(19) United States
(12) Patent Application Publication
(10) Pub. No.: US 2023/0210813 A1
(43) Pub. Date: Jul. 6, 2023

(54) METHODS AND PHARMACEUTICAL COMPOSITIONS FOR THE TREATMENT OF CHRONIC KIDNEY DISEASE

(71) Applicants: INSERM (INSTITUTE NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); UNIVERSITÉ TOULOUSE III - PAUL SABATIER, Toulouse (FR)

(72) Inventors: Joost SCHANSTRA, Toulouse Cedex 4 (FR); Julie KLEIN, Toulouse Cedex 4 (FR); Odile SCHILTZ, Toulouse Cedex 4 (FR); Jean-Loup BASCANDS, Sainte Clotilde - la Reunion (FR)

(21) Appl. No.: 18/001,142

(22) PCT Filed: Jun. 10, 2021
(86) PCT No.: PCT/EP2021/065687

§ 371 Date: Dec. 8, 2022

(30) Foreign Application Priority Data
Jun. 11, 2020 (EP) 20305636.1

(51) Int. Cl. A61K 31/365; A61K 31/403; A61P 13/12

(52) U.S. Cl. CPC A61K 31/365; A61K 31/403; A61P 13/12

(57) ABSTRACT

The present invention relates to methods and pharmaceutical compositions for the treatment of chronic kidney disease. The inventors used the CMap resources to identify the sesquiterpene lactone parthenolide that was subsequently analyzed for its in vivo capacity to reduce the development of DKD in the type I diabetes mouse model. In particular, the invention relates to a method of treating chronic kidney disease (CKD) in patient in need thereof comprising administering to the patient a therapeutically effective amount of Dimethylaminoparthenolide (DMAPT).

### FIELD OF THE INVENTION

The present invention relates to methods and pharmaceutical compositions for the treatment of chronic kidney disease.

### BACKGROUND OF THE INVENTION

On a world scale the total number of individuals with chronic kidney disease (CKD) exceeds 840 million, a truly concerning figure that is >20 times higher than the number of individuals affected by AIDS/HIV worldwide. Mortality from CKD is increasing faster than mortality for most other causes and is expected to become the fifth leading global cause of death by 2040. CKD induced by diabetes, “diabetic kidney disease” (DKD), is the leading cause of CKD.

Diabetic kidney disease (DKD) has a high incidence (30-40%) in diabetic patients and about 50% of patients with DKD develop renal failure in the long term. In both human and animals, the presence of DKD is characterized by increased urinary albumin excretion resulting from apoptosis and functional loss of podocytes, resulting in impairment of glomerular filtration and glomerulosclerosis and, in more advanced DKD stages, tubulo-interstitial fibrosis^{1,2}. Because glomerular injury marks the initial event of DKD, glomeruli are relevant targets to investigate the early molecular mechanisms of DKD pathogenesis.

The therapeutic options for preventing DKD are limited. Over the last decades, blockers of the renin angiotensin aldosterone system (RAAS, i.e. angio-converting enzyme inhibitors (ACEi) and angiotensin receptor blockers) have been the most widespread pharmacological treatments slowing down the development of DKD and its progression. However, 25-30% of the patients still develop DKD³.

Although reducing DKD progression in patients with advanced DKD with macroalbuminuria⁴, RAAS inhibitors (RAASi) are poorly efficient in preventing DKD in patients with microalbuminuria. Moreover, RAAS inhibitors can increase the risk of hyperkalemia when used at high doses or when combining two RAAS blockers⁵.

Therefore, additional therapies or drugs to prevent the progression of DKD are urgently needed. Emerging therapies undergoing clinical trials are focussing on expanding RAAS blockade with double angiotensin receptor/endothelin receptor blockers, antidiabetic drugs with additional nephroprotective properties such as sodium-glucose transporter 2 (SGLT2) inhibitors and glucagon-like peptide-1 (GLP-1) agonists, or targeting inflammation (pentoxifylline, a methylxanthine phosphodiesterase) or transcription factor Nrf2 (bardoxolone)⁶. However, even if successful, drugs used in these trials are focussing on single targets that will impact only part of the complex processes involved in the development of DKD. The complexity of DKD has prompted researchers to move away from investigating known candidate pathways and a focus on single molecules, to unbiased omics based studies⁷. This has resulted in a number of recent omics studies in the field identifying potential biomarkers of DKD⁸, but not necessarily resulting in novel drugs yet. This could be attributed to the fact that potential targets need to be transformed into drugs which often takes a substantial amount of time and effort, and/or, that most studies employed genomic and transcriptomic strategies which not necessarily translate to changes in proteins that are considered as the major targets of drugs⁹.

The inventors adopted a systems biology strategy of drug repurposing based on the analysis of protein signatures aiming to find a new drug that would complement the beneficial effects of the widely used RAAS blockers in DKD treatment. To this end, the inventors established the glomerular protein signature of DKD in a mouse model of type I diabetes treated with and without ACEi that they then compared with a database of thousands of molecular signatures of potential bioactive compounds (Connectivity MAP: https://portals.broadinstitute.org/cmap/). Connectivity Map (CMap) algorithms enable data-driven studies on drug repositioning^{10,11}. The CMap resource allows to compare a query gene signature to a differential gene expression database built by treating human cell lines with a wide range of chemical compounds. CMap analysis outcome is a list of compounds ranked according to their similarity to that of the query gene signature (positive or negative enrichment). CMap has been applied in several studies including by them with goals to identify candidate drugs for repurposing^{12,13} Here, the inventors used CMap resources to identify the sesquiterpene lactone parthenolide that was subsequently analyzed for its in vivo capacity to reduce the development of DKD in the type I diabetes mouse model.

### SUMMARY OF THE INVENTION

The present invention relates to methods and pharmaceutical compositions for the treatment of chronic kidney disease. In particular, the present invention is defined by the claims.

### DETAILED DESCRIPTION OF THE INVENTION

While blocking the renin angiotensin aldosterone system (RAAS) has been the main therapeutic strategy to control diabetic kidney disease (DKD) for many years, 25-30% of diabetic patients still develop the disease. In the present work the inventors adopted a system biology strategy to analyze glomerular protein signatures to identify drugs with potential therapeutic properties in DKD acting through a RAAS-independent mechanism. Glomeruli were isolated from wild type and type 1 diabetic mice (Ins2Akita) treated or not with the angiotensin-converting enzyme inhibitor (ACEi) ramipril. Large scale quantitative proteomics was used to identify the DKD-associated glomerular proteins (DKD-GPs) that were ramipril-insensitive (RI-DKD-GPs). The inventors then applied an in silico drug repurposing approach using a pattern-matching algorithm (Connectivity Mapping) to compare the RI-DKD-GPs's signature with a collection of thousands of transcriptional signatures of bioactive compounds. The sesquiterpene lactone parthelonide was identified as one of the top compounds predicted to reverse the RI-DKD-GPs's signature.

Treatment of diabetic Ins2Akita mice with dimethylaminoparthenolide (DMAPT), a water-soluble analogue of parthenolide, significantly reduced urinary the urinary albumin/creatine ratio (ACR), glomerulosclerosis and tubulointerstitial fibrosis. This contrasts with ramipril treatment which only reduced ACR without modifying DKD-associated renal-injuries.

Using a system biology approach the inventors identified DMAPT, as a compound with a potential add-on value to standard-of-care ACEi-treatment in DKD.

Accordingly, an object of the present invention relates to a method of treating a chronic kidney disease (CKD) in a patient in need thereof comprising administering to the patient a therapeutically effective amount of dimethylaminoparthenolide (DMAPT).

In some embodiment, the present invention relates to dimethylaminoparthenolide (DMAPT) for use in a method of treating a chronic kidney disease (CKD) in a patient in need thereof.

As used herein, the term “subject” denotes a mammal, such as a rodent, a feline, a canine, and a primate. Particularly, the subject according to the invention is a human. As used herein, the term “subject” encompasses “patient”.

In some embodiment, the subject of the present invention is suffering or will suffer from chronic kidney disease (CKD).

As used herein, the term “chronic kidney disease” or “CKD” has its general meaning in the art and refers to a progressive loss in renal function over a period of months or years. CKD is used to classify numerous conditions that affect the kidney, destruction of the renal parenchyma and the loss of functional nephrons or glomeruli. It should be further noted that CKD can result from different causes, but the final consequence remains renal fibrosis. CKD is defined as kidney damage or glomerular filtration rate (GFR)<60 mL/min/1.73 m² for 3 months or more, irrespective of cause. Kidney damage in many kidney diseases can be ascertained by the presence of albuminuria, defined as albumin-to-creatinine ratio >30 mg/g in two of three spot urine specimens. GFR can be estimated from calibrated serum creatinine and estimating equations, such as the Modification of Diet in Renal Disease (MDRD) Study equation or the Cockcroft-Gault formula. Kidney disease severity is classified into five stages according to the level of GFR. Examples of etiology of CKD include, but are not limited to, cardiovascular diseases, hypertension, diabetes, glomerulonephritis, polycystic kidney diseases, and kidney graft rejection. In some, the patient in need thereof suffers from a disease selected from the group consisting of nephropathy (e.g. membranous nephropathy (MN), diabetic nephropathy and hypertensive nephropathy), glomerulonephritis (e.g. membranous glomerulonephritis and membranoproliferative glomerulonephritis (MPGN) such as rapidly progressive glomerulonephritis (RPGN)), interstitial nephritis, lupus nephritis, idiopathic nephrotic syndrome (INS) (e.g. minimal change nephrotic syndrome (MCNS) and focal segmental glomerulosclerosis (FSGS)), obstructive uropathy, polycystic kidney disease (e.g. Autosomal Dominant Polycystic Kidney Disease (ADPKD) and Autosomal Recessive Polycystic Kidney Disease (ARPKD)), cardiovascular diseases, hypertension, diabetes (e.g. diabetic nephropathy), and kidney graft rejection (e.g. acute and chronic kidney rejection).

In some embodiment, the CKD is focal segmental glomerulosclerosis (FSGS).

In some embodiment, the CKD is a progressive CKD after a partial nephrectomy.

In some embodiment, the CKD is a diabetic kidney disease (DKD).

Accordingly, an object of the present invention relates to a method of treating a diabetic kidney disease (DKD) in a patient in need thereof comprising administering to the patient a therapeutically effective amount of dimethylaminoparthenolide (DMAPT).

In some embodiment, the present invention relates to dimethylaminoparthenolide (DMAPT) for use in a method of treating a diabetic kidney disease (DKD) in a patient in need thereof.

As used herein the term “diabetic kidney disease” (DKD) also known as diabetic nephropathy, has its general meaning in the art and refers to a type of kidney disease caused by diabetes. It happens when high blood sugar damages the kidneys. The earliest sign of diabetic kidney disease is an increased excretion of albumin in the urine.

As used herein the term “diabetes” has its general meaning in the art and refers to a chronic disease characterized by the presence of excess blood sugar called hyperglycemia. It is known if the fasting blood sugar level is equal to or greater than 1.26 g/1 or 7 mmol/1 of blood during two successive dosages.

In some embodiment, the subject of the present invention suffering from DKD has type 1 diabetes.

In some embodiment, the subject of the present invention suffering from DKD has type 2 diabetes.

As used herein the term “insulin” has its general meaning in the art and refers to a hormone made by the pancreas, which is permanently present in the blood. Its role is to maintain blood sugar around 1 g/1 when sugar intake is high: insulin is a hypoglycemic hormone. Insulin allows the body's cells to take up the blood sugar when it needs it (such as muscle cells during exercise) and use it to turn it into energy. If necessary, it allows the storage of unused sugar, in the liver or fat cells. When the sugar level rises, for example after a meal, the pancreas produces more insulin to bring the blood sugar level back to normal. If insulin is insufficient or ineffective, sugar builds up in the blood and the blood sugar rises excessively: this is hyperglycemia. In the absence of treatment, this hyperglycemia is maintained at too high a level: it is chronic hyperglycemia which defines diabetes. There are 2 main types of diabetes:
“type 1” diabetes, is an autoimmune disease and is due to an absence of insulin secretion by the pancreas. In its absence, cells can no longer properly use the sugar that circulates in the blood. Hyperglycemia appears quickly, as soon as the insulin level becomes insufficient. The type 1 diabetes most commonly occurs in children, adolescents and young adults.
“type 2” diabetes, caused by the body's improper use of insulin. Its development takes place very gradually, insidiously over many years. First, the body's cells become resistant to insulin. This resistance is normal with age, but it is aggravated by excess fatty tissue in the event of overweight and obesity. This stage is called: insulin resistance. Glucose builds up in the blood and hyperglycemia gradually sets in; the body is trying to adapt. The pancreas increases the production of insulin: this is called hyperinsulinism; after several years (10 to 20 years), the pancreas becomes exhausted and can no longer secrete enough insulin to regulate blood sugar: this is the stage of insulin deficiency.

The present invention relates to a method of preventing or reversing vascular calcification in patients with chronic kidney disease (CKD).

In some embodiment, the subject has a vascular calcification. As used, herein the term “vascular calcification” refers to a mineral deposition, in the vasculature, in a form of calcium-phosphate complexes. Although vascular calcification is regarded as part of the normal aging process, certain pathological processes such as chronic kidney disease (CKD) and diabetes, may precipitate the condition. Vascular calcification is a process characterized by thickening and loss of elasticity of muscular artery walls. Calcification is classified into two forms, depending on where the mineral is deposited. Indeed, this thickening and loss of elasticity occurs in two distinct sites, the intimal and medial layers of the vasculature. Intimal calcification is closely related to lipid deposits, and the clinically relevant infiltration of inflammatory cells, with obstructive arterial disease, whereas the latter is more pronounced by transformation into osteoblast-like cells from smooth muscle cells. Intimal calcification associated with atherosclerosis is present in the general population and accelerated by CKD. Medial calcification is characteristic of CKD, up to 45 fold more prevalent than in individuals without CKD, but is also increased in diabetes and aging.

In some embodiment, the vascular calcification is an intimal calcification or a medial calcification.

As used herein the term “parthenolide” has its general meaning in the art and refers to a sesquiterpene lactone of the germacranolide class which occurs naturally in the plant feverfew (Tanacetum parthenium), a member of the Asteraceae family. It is found in highest concentration in the flowers and fruit. The parthenolide has also the following International Union of Pure and Applied Chemistry (IUPAC) name (1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one and the following of formula:

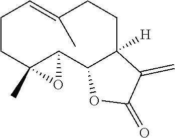 [SMILES: C=C1C(=O)O[C@@H]2[C@H]3O[C@]3(C)CC/C=C(\C)CC[C@@H]12]

As used herein the term “parthenolide derivatives” refers to the derivatives of parthenolide. In embodiments the parthenolide derivative is selected from the group comprising 8-, 9- or 14-hydroxy parthenolide and/or 13-amino parthenolides such as 13-dimethylamino parthenolide usually referred to as dimethylamino parthenolide (DMAPT). Hydroxy derivatives may be selected from the group comprising 8-, 9- or 14-hydroxy parthenolide, particularly hydroxy-8a-parthenolide. 13-amino parthenolide derivatives may be selected from the group comprising 11 βH, 13-Dimethylaminoparthenolide, 11βH, 13-Diethylaminoparthenolide 11βH, 13-(tert-Butylamino) parthenolide, 11βH, 13-(Pyrrolidin-1-yl) parthenolide, 11βH, 3-(Piperidin-1-yl) parthenolide, 11βH, 13-(Morpholin-1-yl)parthenolide, 11 PH, 13-(4-Methylpiperidin-1-yl) parthenolide, 11βH, 13-(4-Methylpiperazin-1-yl) parthenolide, 11βH, 13-(Homopiperidin-1-yl) parthenolide, 11βH, 13-(Heptamethyleneimin-1-yl) parthenolide, 11βH, 13-(Azetidin-1-yl) parthenolide and/or 11βH, 13-Diallylaminoparthenolide. In some embodiment, the parthenolide derivatives is dimethylamino parthenolide (DMAPT).

As used herein the term “dimethylaminoparthenolide” (DMAPT), also known as 13-dimethylamino parthenolide, has its general meaning in the art and refers to a water-soluble parthenolide. DMAPT has also the following International Union of Pure and Applied Chemistry name (4E,8S)-8-[(dimethylamino)methyl]-2,3,6,7,7aS,8,10aS,10bR-octahydro-1aR,5-dimethyl-oxireno[9,10]cyclodeca[1,2-b]furan-9(1aH)-one and the following formula:

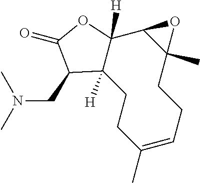

As used herein, the term “treatment” or “treat” refer to both prophylactic or preventive treatment as well as curative, improving the patient's condition or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical deficit or who ultimately may acquire the deficit, in order to prevent, cure, delay the onset of, reduce the severity of, or ameliorate one or more symptoms of a deficit or recurring deficit, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., daily, weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “preventing” intends characterizing a prophylactic method or process that is aimed at delaying or preventing the onset of a deficit or condition to which such term applies.

As used herein the terms “administering” or “administration” refer to the act of injecting or otherwise physically delivering a substance as it exists outside the body (e.g. DMAPT or RAAS inhibitor) into the subject, such as by oral, mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the DMAPT or RAASi are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1,2, 3,4, 5,6,7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Typically, the DMAPT or the RAAS inhibitor as described above are administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The present invention also provides for therapeutic applications where the DMAPT of the present invention is used in combination with at least one further therapeutic agent, e.g. for treating CKD. Such administration may be simultaneous, separate or sequential. For simultaneous administration the agents may be administered as one composition or as separate compositions, as appropriate. The further therapeutic agent is typically relevant for the deficit to be treated.

In some embodiments, the targeted therapy consists of administering to the subject DMAPT in combination with any nephroprotective treatment.

As used herein the term “nephroprotective treatment” refers to treatment tending to preserve kidney function especially when the kidneys are exposed to unusual or unique stresses.

In some embodiments, the nephroprotective treatment includes but are not limited to RAAS inhibitor (RAASi), sodium-glucose cotransporter 2 inhibitor (SGLT2i), anti-endothelin-1 receptor . . . .

In some embodiments, the targeted therapy consists of administering to the subject DMAPT in combination with an anti-endothelin-1 receptor.

In a particular embodiment, i) DMAPT and ii) an anti-endothelin-1 receptor as a combined preparation according to the invention for simultaneous, separate or sequential use in the method for treating CKD (e.g. DKD) in a subject.

As used herein the term “endothelin receptor” is composed of at least four receptors ET_{A}, ET_{B1}, ET_{B2} and ET_{C}.

The endothelin receptor type A (ET_{A}) is a subtype for vasoconstriction. These receptors are found in the smooth muscle tissue of blood vessels, and binding of endothelin to ET_{A} increases vasoconstriction (contraction of the blood vessel walls) and the retention of sodium, leading to increased blood pressure. The endothelin receptor type B1 (ET_{B1}) mediates vasodilation. When endothelin binds to ET_{B1} receptors, this leads to the release of nitric oxide (also called endothelium-derived relaxing factor), natriuresis and diuresis (the production and elimination of urine) and mechanisms that lower blood pressure. The endothelin receptor type B2 (ET_{B2}) mediates vasoconstriction. The endothelin receptor type C (ET_{C}) has yet no clearly defined function.

As used herein the term “anti-endothelin-1 receptor” refers to a drug that blocks endothelin receptors. Three main kinds of anti-endothelin-1 receptor exist:
selective receptor antagonists which include but are not limited to sitaxentan, ambrisentan, atrasentan, BQ-123, zibotenta, sparsentesan. They affect endothelin A receptors.
dual antagonists which include but are not limited to bosentan, macitentan, tezosentan . . . They affect both endothelin A and B receptors.
selective receptor antagonists which include but are not limited to BQ-788 and A192621 . . . They affect endothelin B receptors.

In some embodiments, the targeted therapy consists of administering to the subject DMAPT in combination with a sodium-glucose cotransporter 2 inhibitor (SGLT2i).

In a particular embodiment, i) DMAPT and ii) a SGLT2i as a combined preparation according to the invention for simultaneous, separate or sequential use in the method for treating CKD (e.g. DKD) in a subject.

As used herein the term “sodium-glucose cotransporter 2 inhibitor” (SGLT2i) also called gliflozins, refers to a class of medications that inhibit reabsorption of glucose in the kidney and therefore lower blood sugar. SGLT2i acts by inhibiting sodium-glucose transport protein 2 (SGLT2). Examples of SGLT2i include but are not limited to canagliflozin, dapagliflozin, empagliflozin, ertugliflozin, ipragliflozin, luseogliflozin, remogliflozin etabonate, sergliflozin etabonate, sotagliflozin, tofogliflozin.

In some embodiments, the targeted therapy consists of administering to the subject DMAPT in combination with a RAAS inhibitor (RAASi).

In a particular embodiment, i) DMAPT and ii) a RAASi as a combined preparation according to the invention for simultaneous, separate or sequential use in the method for treating CKD (e.g. DKD) in a subject.

As used herein the term “renin angiotensin system” (RAS) also known as “renin-angiotensin-aldosterone system” (RAAS) has its general meaning in the art and refers to a hormone system that regulates blood pressure and fluid and electrolyte balance, as well as systemic vascular resistance. The first stage of the RAAS is the release into the blood of the enzyme renin. Angiotensinogen is a precursor protein produced in the liver and cleaved by renin to form angiotensin I. Angiotensin I is then converted to angiotensin II by angiotensin converting enzyme (ACE). Finally, angiotensin II acts on the adrenal cortex to stimulate the release of aldosterone. Aldosterone is a mineralocorticoid, a steroid hormone released from the zona glomerulosa of the adrenal cortex.

As used herein, the term “inhibitor” as used herein includes not only drugs for inhibiting activity of target molecules, but also drugs for inhibiting the expression of target molecules.

As used herein the term “renin-angiotensin-aldosterone system inhibitors” (RAASi or RAAS inhibitor) has its general meaning in the art and refers to a group of drugs that act by inhibiting the renin-angiotensin-aldosterone system (RAAS) and include angiotensin-converting enzyme (ACE) inhibitors, angiotensin-receptor blockers (ARBs), and direct renin inhibitors.

Examples of ACE inhibitors (ACEi) include but are not limited to Enalapril, lisinopril, ramipril, captopril, benazepril. Examples of ARBs include but are not limited to Valsartan, candesartan, losartan, irbesartan. Example of direct renin inhibitors include but are not limited to Aliskiren.

In some embodiments, the targeted therapy consists of administering to the subject DMAPT in combination with ACE inhibitors (ACEi).

In a particular embodiment, i) DMAPT and ii) a ACEi as a combined preparation according to the invention for simultaneous, separate or sequential use in the method for treating CKD (e.g. DKD) in a subject.

In some embodiments, the targeted therapy consists of administering to the subject DMAPT in combination with ramipril.

In a particular embodiment, i) DMAPT and ii) ramipril as a combined preparation according to the invention for simultaneous, separate or sequential use in the method for treating CKD (e.g. DKD) in a subject.

In some embodiments, the targeted therapy consists of administering to the subject DMAPT in combination with angiotensin-receptor blockers (ARBs).

In a particular embodiment, i) DMAPT and ii) a ARBs as a combined preparation according to the invention for simultaneous, separate or sequential use in the method for treating CKD (e.g. DKD) in a subject.

As used herein, the term “combination” is intended to refer to all forms of administration that provide a first drug together with a further (second, third . . . ) drug. The drugs may be administered simultaneously, separately or sequentially and in any order. According to the invention, the drug is administered to the subject using any suitable method that enables the drug to reach the kidney. In some embodiments, the drug administered to the subject systemically (i.e. via systemic administration). Thus, in some embodiments, the drug is administered to the subject such that it enters the circulatory system and is distributed throughout the body.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

The DMAPT or the RAAS inhibitor as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The invention will be further illustrated by the following figure and example. However, these examples and figures should not be interpreted in any way as limiting the scope of the present invention.

### FIGURE

FIG. 1: Comparative influence of DMAPT and Ramipril-treatment on Ins2Akita mice. 4 month old diabetic Ins2Akita (DKD) that had been treated or not (WT) with Ramipril (DKD+R) or DMAPT (DKD+D) for 2 months before sacrifice (scale bar=50 μm). (A-F) Quantification of ACR (A), glomerular injury (B), glomerular area (C), fibrosis (D), glycemia (E), and body weight (F) in WT (n=10), DKD (n=9), DKD+R (n=9) and DKD+D (n=9). Values are mean±SEM and One-way ANOVA test for multiple comparisons. Comparison to WT: *: P<0.05; **:P<0.01; ***: P<0.001; ****: P<0.0001. Comparison of DKD+R or DKD+D to DKD: #: P<0.05; ##:P<0.01; ###: P<0.001; ####: P<0.0001. Comparison of DKD+R to DKD+D: @@:P<0.01; @@@@: P<0.0001.

### EXAMPLE

Material & Methods

Animal

Male C57BL/6-Ins2Akita/J (Ins2Akita) and C57BL/6J (WT) mice (Charles River) were housed with unrestricted access to water and were maintained on a 12-h light-dark cycle in a pathogen-free environment on standard mouse chow. All experiments were conducted in accordance with the Guide for the care and use of laboratory animals of the National Institute of Health, eighth edition and the French Institute of Health guidelines for the care and use of laboratory animals. The project was approved by the local (Inserm/UPS US006 CREFRE) and national ethics committees (ethics committee for animal experiment, CEEA122; Toulouse, France; approval 02867.01).

Treatments and Urine Collection

In a first series of experiments, 2 months old mice were treated with or without ACEi ramipril (10 mg/kg/d in drinking water) for 2 months. In a second series of experiments 2 months old mice were treated with or without ACEi ramipril as in the first series or with dimethylaminoparthenolide monofumarate (DMAPT, 10 mg/kg/d by gavage) for 2 months. In each series of experiments, urine was collected in metabolic cages for 12 h, few days before sacrifice.

Isolation of Glomeruli

Glomeruli were isolated as previously described³¹ with minor modifications. Mice were anesthetized by ip injection of a mixture of ketamine (100 mg/kg) and xylazine (10 mg/kg) in phosphate buffered saline (PBS). A catheter was placed into the abdominal aorta after ligation of the cava vein, the upper abdominal aorta, the mesenteric and the celiac arteries. The lower part of the abdominal aorta was perfused with 40 ml of Dynabeads M-450 Tosylactivated (4.5 μm diameter, Dynal A.S., Oslo, Norway) at a concentration of 2.106 beads/ml followed by 15 ml of cold PBS. This procedures allows accumulation of beads in glomeruli³². Next, the left and right kidneys were collected and decapsulated. The animal was sacrificed by cervical dislocation and blood was collected by intra-cardiac puncture in heparinized tubes and plasma was prepared by centrifugation at 1500×g at 4° C. for 5 min and stored at −20° C. for further use. One portion of the right kidney was snap-frozen in liquid nitrogen and stored at −80° C. Another portion of the right kidney cortex was fixed in Carnoy's solution (ethanol/chloroform/glacial acetic acid: 60/30/10, v/v/v) for further histological analysis.

The left kidney was gently pressed manually through a 70 μm cell strainer using a flattened pestle followed by washing of the cell strainer with 20 ml of cold PBS. The filtrate was centrifuged at 200×g for 5 minutes at 4° C., and the glomerular pellet was adjusted to 2 ml with PBS and transferred to an Eppendorf tube that was placed in a magnetic particle concentrator (Dynal A.S., Oslo, Norway) to concentrate the glomeruli into a pellet. The supernatant was discarded and the pellet was washed 5×with 1 ml of PBS. The final pellet was resuspended in 100 μl PBS. This procedure allows the isolation of ˜4000 glomeruli per kidney.

Based on light microscopy survey, our glomerular suspensions were highly enriched for glomeruli. Moreover, mRNA quantification showed that isolated glomeruli were enriched in glomerular-specific genes (nphs2, podx1 and cldn5) while proximal (aqp1, slc22a13), distal tubules (wnk), and of loop of Henle (aqp1, slcl2a3) specific genes were not, or poorly enriched compared to total kidney. Moreover, among the 2422 proteins detected by LC-MS/MS, several glomerular-specific proteins were ranked within the first upper quartile of relative abundance including NphS1 (rank 196), Tjpl/ZO-1 (rank 221) Nphs2 (rank 339), Synpo (rank 388), Actn4 (rank 7), cd2ap (rank 543), while several tubular-specific proteins Aqp1 (rank 591), Umod (rank 1634) were ranked as less abundant proteins, or were not detected (slc22a13, Wnk). These observations support the relative high purity of our glomerular preparations.

Renal Histology

Right kidney cortexes were fixed in Carnoy's solution for 24 h, dehydrated in successive baths with increasing alcohol concentrations, embedded in paraffin and 2 μm sections were cut and mounted, and stained with either periodic acid-Schiff (PAS) or Masson trichrome. Stained sections were scanned using a Nanozoomer 2.0 RS (Hamamatsu Photonics SARL) and treated with the Morpho-expert image-analysis software (version 1.00, Explora Nova) for renal corpuscle surface quantification. At least 50 glomeruli including superficial and juxtaglomerular cortical area, were examined for each animal. The extent of glomerular injury was expressed as the percentage of glomerular area fraction occupied by PAS positive matrix³.

Biochemical Analysis

Urinary albumin concentration was measured by ELISA using the AlbuWell kit (WAK-Chemie Medical GmbH, Steinbach, Germany). Urinary creatinine concentration was measured by the colorimetric method of Jaffe according to the protocol Creatinine Assay Kit (Bio Assay Systems). Blood glucose levels were measured in caudal blood from fasted awake mice using a glucometer (Glucometer Elite XL; Bayer Healthcare, Elkhart, Ind.).

Quantitative Proteomics of Glomerular Samples

Glomerular sample preparation for proteomics—Isolated glomeruli were homogenized in RIPA buffer under agitation for 3 min and centrifuged 15 min at 13000×g to pellet the beads together with cell debris. The supernatant was collected and stored at −80° C. at a protein concentration of 1-2 mg/ml before being processed for mass spectrometry (MS) analysis. Protein samples were air-dried in a SpeedVac concentrator and then reconstituted in 1× final Laemmli buffer containing 25 mM dithiothreitol and heated at 95° C. for 5 min. Cysteines were alkylated for 30 min at room temperature by the addition of a solution of 75 mM chloroacetamid. Proteins were loaded onto a 12% acrylamide SDS-PAGE gel and concentrated in a single band visualized by Coomassie staining (Instant Blue—Expedeon). The gel band containing the whole sample was cut and washed several times in 50 mM ammonium bicarbonate:acetonitrile (1:1) for 15 min at 37° C. Proteins were in-gel digested using 0.6 μg of modified sequencing-grade trypsin (Promega) in 50 mM ammonium bicarbonate overnight at 37° C. Peptides were extracted from the gel by two incubations in 10% formic acid:acetonitrile (1:1) for 15 min at 37° C. The extracted fractions were pooled with the initial digestion supernatant and dried under speed-vaccum. The resulting peptides were resuspended with 14 μL of 5% acetonitrile, 0.05% trifluoroacetic acid for nanoLC-MS/MS analysis.

NanoLC-MSMS analysis—Peptides were analyzed by nanoLC-MS/MS using an UltiMate 3000 system (Dionex) coupled to an LTQ Orbitrap Velos ETD mass spectrometer (Thermo Fisher Scientific). Five microliters of each sample were loaded onto a C18 precolumn (300 μm inner diameter×5 mm; Dionex) at 20 μl/min in 5% acetonitrile, 0.05% trifluoroacetic acid. After 5 min of desalting, the precolumn was switched online with the analytical C18 column ((75 m inner diameter×50 cm; in-house packed with Reprosil C18) equilibrated in 95% solvent A (5% acetonitrile, 0.2% formic acid) and 5% solvent B (80% acetonitrile, 0.2% formic acid). Peptides were eluted using a 5 to 50% gradient of solvent B over 110 min at a flow rate of 300 nl/min. The mass spectrometer was operated in a data-dependent acquisition mode with Xcalibur software. Survey MS scans were acquired in the Orbitrap on the 300 to 2000 m/z range with the resolution set at 60,000. The 20 most intense ions per survey scan were selected for CID fragmentation and the resulting fragments were analyzed in the linear ion trap (LTQ). A dynamic exclusion of 60 s was used to prevent repetitive selection of the same peptide. Each sample was injected once for MS analysis.

Protein identification and quantifcationfrom raw nanoLC-MS/MS data—Raw nanoLC-MS/MS files were processed with the MaxQuant software (version 1.5.2.8) for database search with the Andromeda search engine and for quantitative analysis. Data were searched against “Mus musculus” entries in the Swiss-Prot protein database (UniProtKB/Swiss-Prot protein knowledgebase release 2015_01; 16,695 entries). Carbamidomethylation of cysteine was set as a fixed modification whereas oxidation of methionine and protein N-terminal acetylation were set as variable modifications. Specificity of trypsin digestion was set for cleavage after K or R and two missed trypsin cleavage sites were allowed. The precursor mass tolerance was set to 20 ppm for the first search and 4.5 ppm for the main Andromeda database search. The mass tolerance in MS/MS mode was set to 0.8 Da. Minimum peptide length was set to 7 amino acids and minimum number of unique peptides was set to 1. Andromeda results were validated by the target-decoy approach using a reverse database at both a peptide and protein FDR of 1%. For label-free relative quantification of the samples, the “match between runs” option of MaxQuant was enabled with a time window of 3 min to allow cross-assignment of MS features detected in the different runs.

Data processing and statistical analysis—Protein entries identified as potential contaminants from the ‘proteinGroups.txt’ files generated by MaxQuant were eliminated from the analysis, as were proteins identified by fewer than two peptides. Protein relative quantification was performed by comparisons of different groups of eight samples each (8 biological replicates per group: WT, DKD, DKD+R, WT+R) (Table S1). Protein intensities were normalized across all conditions by the median intensity. For each comparison, only proteins which were quantified in at least 4 biological replicates (4 intensities values retrieved by MaxQuant) in at least one of the groups were considered for further processing and statistical analysis (Filter 1, columns AR to AU, Table S1). Remaining missing values were then replaced by a constant noise value determined independently for each analytical run as the 1% percentile of the total protein population. The mean proportion of missing values over the whole analytic run was 2.1% (line 2435, column S, Table S1). Proteins with a p-value of less than 0.05 were considered as significantly varying between two groups.

Proteomic Data Availability

Because of the confinement resulting COVID-19 pandemic, access to our MS facility was not possible before submission and the proteomic raw data can therefore not be deposited at the time of manuscript submission. Once the pandemic is over, the deposit will be made immediately on ProteomeXchange and the accession number will be communicated.

Pathway Enrichment Analysis

Pathway enrichment analysis of up- and down-regulated proteins was using the Gene Set Enrichement Analysis (GSEA) software package (https://www.gsea-msigdb.org)^{10,34} using “Hallmarks gene sets” and “Canonical Pathways” as Compute Overlaps.

Connectivity Map Analysis

The initial version of CMap (CMap1: https://portals.broadinstitute.org/cmap) consists of 6,100 differential expression profiles obtained after treatment of 3 cultured human cells (MCF7, PC3, and HL60) with varying concentrations of 1309 compounds¹¹. More recently, a new CMap version was released³⁵ (CMap2: https://clue.io/) encompassing 8549 differential expression profiles obtained after treatment of 9 cultured human cells (VCAP, A375, A549, HAE1, HCC515, HEPG2, HT29, MCF7, PC3) with varying concentrations of 2428 compounds. For our experiments, each mouse protein ID was first converted to its human ortholog and then converted into human gene ID. Up- and down-gene IDs were then queried to CMap1 and CMap2 to retrieve compounds with best negative enrichment as recently recommended¹⁷.

DMAPT Synthesis

Dimethylaminoparthenolide monofumarate [(13-(N,N-dimethyl)-amino-4a,5b-epoxy-4,10-dimethyl-6a-hydroxy-12-oic acid-c-lactonegermacra-1(10)-ene monofumarate)] was synthesized by reaction of parthenolide (Sigma-Aldrich) with dimethylamine (Sigma-Aldrich) and isolated as the fumarate salt as previously described¹⁹. Analytical data (1H and 13C NMR, mass spectrometry and melting point) are consistent to those previously reported¹⁶. DMAPT fumarate purity was checked by elemental analysis.

Statistics

Comparison between 2 groups of values was implemented using a two-tailed unpaired Welch's t-tests. Comparison between more than 2 groups, was implemented using an ordinary one-way ANOVA followed by Homl-Sidak's multiple comparisons test was used. P<0.05 was considered statistically significant. For the proteomic data the P values were adjusted for the false discovery rate (Benjamini-Hochberg).

Results

Influence of ramipril on diabetic nephropathy.

Our investigations were performed in the Ins2Akita mouse since this model at the timepoints investigated is recognized as a useful model of early to moderately advanced renal morphological changes and renal dysfunction in type I DKD 14. Moreover, it was shown that ACEi reduces albuminuria in this model^{15,16} Ins2Akita mice (DKD) became significantly hyperglycemic at 1 month of age (Data not shown) and exhibited significant increased ACR at 2 months (Data not shown) compared to WT mice. Ramipril treatment, starting at 2 months of age, of Ins2Akita mice (DKD+R) significantly reduced ACR that reached a level close to that of WT mice (Data not shown) Ramipril also slightly but significantly reduced glycemia (Data not shown) but had no influence on body weight (Data not shown). These data confirmed the ability of ramipril to reduce albuminuria in Ins2Akita mice.

Identification of Ramipril-Sensitive and -Insensitive Glomerular Proteins.

Since our objective was to identify a drug able to inhibit DKD acting through a RAAS-independent mechanism, and since, glomerular injury is considered as the initial key event in DKD progression, we aimed to identify glomerular proteins (GPs) modified in DKD but not counter regulated by ramipril treatment. We hypothesized that such proteins could be potential valuable targets for new pharmacological treatments of DKD on top of ACEi. High quality glomeruli were isolated (see Methods) from each mouse of 4 different groups: WT, DKD, DKD+R and WT+R (8 mice per group) and GPs of each animal were separately analyzed by large-scale quantitative MS-based proteomics.

Based on the analysis of 32 glomerular samples (4 groups of 8 animals), a total of 2422 GPs were identified and quantified (Data not shown). Three sets of comparison (Set #1, Set #2, Set #3) were then performed (Data not shown). Proteins were considered significantly different in those comparisons based on a p<0.05. In Set #1, comparison of DKD vs WT mice identified 666 GPs with significantly varying abundances [329up with increased and 337down with decreased abundance] that we considered as DKD-associated and that we entitled DKD-GPs (DKD-associated glomerular proteins) (Data not shown). In Set #2, comparison of DKD+R vs DKD mice and identified 543 significant proteins (316 up, 227 down) corresponding to ramipril-sensitive GPs (RS-GPs), and 1879 not significant proteins corresponding to ramipril-insensitive GPs (RI-GPs) in a DKD context (Data not shown). Finally, in Set #3 comparison of WT+R vs WT mice identified 500 (252 up, 248 down) significant RS-GPs, and 1922 not significant RI-GPs in a non-DKD context (Data not shown).

We then classified the 666 DKD-GPs identified in Set #1 in Set #2 and Set #3 (Data not shown). 86 of the 666 DKD-GPs in Set #1 were significant in Set #2 with a ratio opposite to Set #1 (Data not shown) and were classified as RS-DKD-GPs indicating their ability to be counter regulated by ramipril. Nevertheless, in Set #3, 3 of these 86 DKD-GPs showed a regulation opposite to Set #1 (Data not shown) indicating a sensitivity to ramipril not specific to DKD. These 3 proteins were therefore removed from the definitive RS-DKD-GPs list that finally included 83 proteins (35 up, 48 down) (Data not shown). Pathway analysis of these RS-DKD-GPs showed a highly significant enrichment in proteins involved in small molecules transport and in folding of actin and tubulin by the CCT-TRIC complex (Data not shown).

In contrast, 518 DKD-GPs were not significant in Set #2 (Data not shown) and were classified as RI-DKD-GPs indicating their insensitivity to ramipril in a DKD context. Nevertheless, 168 of them were significant in Set #3 (Data not shown), indicating their sensitivity to ramipril in a non-diabetic context. Since our objective was to identify a ramipril-insensitive signature specific to DKD, these 168 proteins were removed from the RI-DKD-GPs list that finally included 346 proteins (173 up, 173 down) (Data not shown). RI-DKD-GPs included 48 proteins with a more than 2-fold increase abudance (Data not shown), and 36 proteins with a more than 2 fold decreased abundance (Data not shown). RI-DKD-GPs represented 52% of all DKD-GPs indicating ample space for improvement of DKD treatment. Pathway analysis of RI-DKD-GPs showed a highly significant enrichment in proteins involved in the metabolism of the amino acids, protein localization and peroxisomal protein import (Data not shown) suggesting that RI-DKD-GPs are involved in quite different molecular pathways than RS-DKD-GPs. Overall these data suggested that RI-DKD-GPs are potential targets for new pharmacological treatments of DKD.

Connectivity Mapping of RI-DKD-GPs Signature.

In an attempt to identify a pharmacological compound able to inhibit DKD through a ramipril-independent mechanism, UP and DOWN RI-DKD-GPs were analyzed in silico using both CMap1 and CMap2 as recommended by Lim and Pavlidis¹⁷ to select to most probable candidates using this in-silico strategy (Methods). Using CMap1, we found 2 top compounds (quizapine and parthenolide) that exhibited the highest negative enrichment score with best “percent non-nul” (100) (Data not shown). Quizapine is a serotonin receptor agonist. Parthenolide is a sesquiterpene lactone naturally present in a plant (Tanacetum parthenium)¹⁸. When using CMap2, quizapine was not retrieved, but parthenolide was found within the top 20 compounds with highest negative enrichment (Data not shown). These observations suggested that parthenolide has the potential to inhibit the ramipril-insensitive glomerular DKD protein signature and therefore the DKD phenotype.

Influence of Parthenolide on DKD.

Following CMap prediction, we decided to verify the capacity of parthenolide to inhibit DKD. Nevertheless, parthenolide has a poor water-solubility that constitutes a major limitation for in vivo studies and for further development as a clinical therapeutic agent. To circumvent this issue, an orally bioavailable analog of parthenolide, DMAPT (dimethyamino-parthenolide, fumarate salt) was developed (supplementary materials and^{19,20}) We therefore tested the influence of a DMAPT-treatment comparatively to ramipril on the development of DKD in Ins2Akita mice.

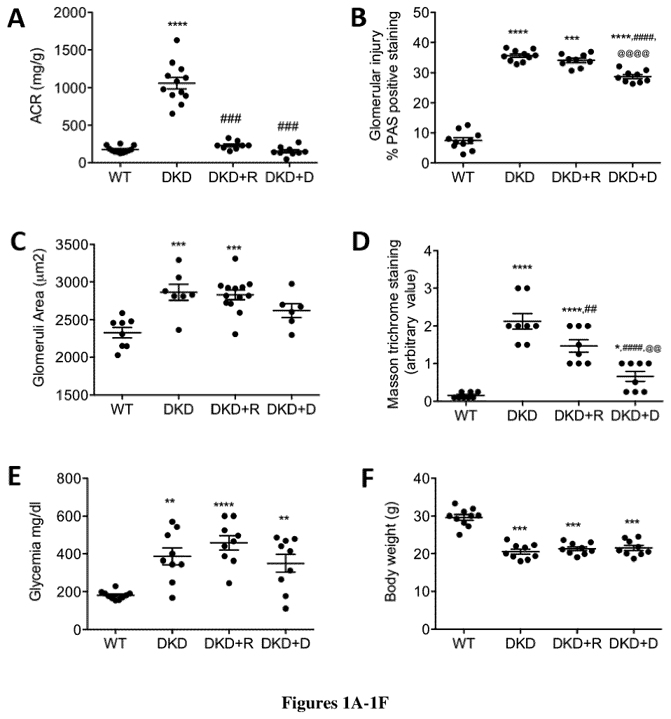

Treatment of Ins2Akita mice (DKD) with DMAPT (DKD+P) led to a significant reduction of ACR to the same extent to that of ramipril (DKD+R) (FIG. 1A). DMAPT also significantly reduced glomerular injury (PAS staining) (FIG. 1B) and fibrosis (Masson trichrome staining) (FIG. 1D), and tended to reduce glomerular area without reaching significance (FIG. 1C). In contrast, ramipril had no significant influence on these 3 parameters (FIG. 1A-FIG. 1D). Neither glycemia (FIG. 1E) nor body weight (FIG. 1F) were significantly influenced by DMAPT and ramipril. For ramipril this contrasts with the slight reduction in glycemia observed in the first experiment (FIG. 1A). In conclusion, these data indicated that DMAPT inhibits DKD-associated albuminuria and that, in contrast to ramipril, DMAPT is also able to inhibit DKD-associated kidney injuries.

Discussion

Because of their beneficial effect on high blood pressure, ACEi reduce cardiovascular risk and CKD progression in patients with advanced DKD with macroalbuminuria, but are poorly efficient in preventing DKD patients with microalbuminuria⁴. Here, we found that among the glomerular proteins that are modified during DKD in Ins2Akita mouse, a model of moderately advanced type I DKD, only a small proportion (12%) is counter regulated by the ACEi ramipril. The remaining insensitive to ramipril proteins are potential targets for new drug-treatment of DKD through a ramipril independent mechanism. By browsing the “drug repurposing” data base CMap, we found that the best negative enrichment of the ramipril-insensitive glomerular DKD protein signature is obtained with parthenolide predicting that this molecule could potentially inhibit DKD progression. Very interestingly, in vivo treatment with DMAPT, an orally bioavailable analog of parthenolide^{19,20} potently reduces urinary ACR in Ins2Akita mice demonstrating that the in silico prediction with CMap was transferable in vivo. Parthenolide was shown to have a beneficial impact on proteinuria and renal injury in immune glomerulonephritis in rat²¹, but to the best of our knowledge the beneficial impact of parthenolide on DKD has not been reported yet. Moreover, our results show that DMAPT is not only able to reduce urinary ACR but is also able to reduce kidney lesions associated with DKD in Ins2Akita mice. This is contrasting with the absence of effects of ramipril-treatment on kidney lesions seen in our and other studiesis^{15,16}. Parthenolide is a sesquiterpene lactone naturally present in a plant (Tanacetum parthenium) that has anti-cancer and anti-inflammatory effects by inhibiting the activity of the NF kappa B transcription factor complex¹⁸. Therefore, the beneficial impact of DMAPT on kidney injuries could depend on the NF kappa B dependent pathways. The hypothesis is in agreement with previous reports showing that dysregulation of NF kappa B is involved in podocyte damage and proteinuria in DKD²²⁻²⁶. The hypothesis is also in agreement with a previous report showing that parthenolide is also able to alleviate renal inflammation and insulin resistance in type 2 diabetic db/db mice²⁷. DMAPT was also reported to inhibit histone deacetylase (HDAC) activity and this effect is independent of NF kappa B²⁸ and there are numerous evidences for renoprotective effects of HDAC inhibitors in experimental DKD²⁹. Therefore, the protective effect of DMAPT in DKD could also result from HDAC inhibition.

Although further studies should investigate a complementary protective effect of ACEi and DMAPT in DKD (in both type I and II diabetes), our data strongly suggested that parthenolide or its derivatives stand as potential new drug candidates for DKD treatment that would advantageously complement the use of ACEi. Phase I trial with standardized doses in patients with cancer showed that parthenolide was well tolerated without dose-limiting toxicity³⁰. Whether parthenolide could be used in patients with DKD remains to be tested.

### REFERENCES

Throughout this application, various references describe the state of the art to which this invention pertains. The disclosures of these references are hereby incorporated by reference into the present disclosure.
1. Tung, C.-W., Hsu, Y.-C., Shih, Y.-H., Chang, P.-J. & Lin, C.-L. Glomerular mesangial cell and podocyte injuries in diabetic nephropathy. Nephrology (Carlton) 23 Suppl 4, 32-37 (2018).
2. Zeni, L., Norden, A. G. W., Cancarini, G. & Unwin, R. J. A more tubulocentric view of diabetic kidney disease. J. Nephrol. 30, 701-717 (2017).
3. Lewis, E. J., Hunsicker, L. G., Bain, R. P. & Rohde, R. D. The effect of angiotensin-converting-enzyme inhibition on diabetic nephropathy. The Collaborative Study Group. N. Engl. J. Med. 329, 1456-1462 (1993).
4. Majewski, C. & Bakris, G. L. Has RAAS Blockade Reached Its Limits in the Treatment of Diabetic Nephropathy? Curr. Diab. Rep. 16, 24 (2016).
5. Yamout, H., Lazich, I. & Bakris, G. L. Blood pressure, hypertension, RAAS blockade, and drug therapy in diabetic kidney disease. Adv Chronic Kidney Dis 21, 281-286 (2014).
6. Ramos, A. M. et al. Design and optimization strategies for the development of new drugs that treat chronic kidney disease. Expert Opin Drug Discov 15, 101-115 (2020).
7. Ruegg, C., Tissot, J.-D., Farmer, P. & Mariotti, A. Omics meets hypothesis-driven research. Partnership for innovative discoveries in vascular biology and angiogenesis. Thromb. Haemost. 100, 738-746 (2008).
8. Colhoun, H. M. & Marcovecchio, M. L. Biomarkers of diabetic kidney disease. Diabetologia 61, 996-1011 (2018).
9. Mokou, M., Lygirou, V., Vlahou, A. & Mischak, H. Proteomics in cardiovascular disease: recent progress and clinical implication and implementation. Expert Rev Proteomics 14, 117-136 (2017).
10. Subramanian, A. et al. Gene set enrichment analysis: a knowledge-based approach for interpreting genome-wide expression profiles. Proc. Natl. Acad. Sci. U.S.A. 102, 15545-15550 (2005).
11. Lamb, J. et al. The Connectivity Map: using gene-expression signatures to connect small molecules, genes, and disease. Science 313, 1929-1935 (2006).
12. Musa, A. et al. A review of connectivity map and computational approaches in pharmacogenomics. Brief. Bioinformatics 19, 506-523 (2018).
13. Schanstra, J. P. et al. Systems biology identifies cytosolic PLA2 as a target in vascular calcification treatment. JCI Insight 4, (2019).
14. Kitada, M., Ogura, Y. & Koya, D. Rodent models of diabetic nephropathy: their utility and limitations. Int J Nephrol Renovasc Dis 9, 279-290 (2016).
15. Lo, C.-S. et al. Dual RAS blockade normalizes angiotensin-converting enzyme-2 expression and prevents hypertension and tubular apoptosis in Akita angiotensinogen-transgenic mice. Am. J. Physiol. Renal Physiol. 302, F840-852 (2012).
16. You, H., Gao, T., Cooper, T. K., Morris, S. M. & Awad, A. S. Arginase inhibition: a new treatment for preventing progression of established diabetic nephropathy. Am. J. Physiol. Renal Physiol. 309, F447-455 (2015).
17. Lim, N. & Pavlidis, P. Evaluation of Connectivity Map shows limited reproducibility in drug repositioning. bioRxiv 845693 (2019) doi:10.1101/845693.
18. Ghantous, A., Sinjab, A., Herceg, Z. & Darwiche, N. Parthenolide: from plant shoots to cancer roots. Drug Discov. Today 18, 894-905 (2013).
19. Neelakantan, S., Nasim, S., Guzman, M. L., Jordan, C. T. & Crooks, P. A. Aminoparthenolides as novel anti-leukemic agents: Discovery of the NF-kappaB inhibitor, DMAPT (LC-1). Bioorg. Med. Chem. Lett. 19, 4346-4349 (2009).
20. Guzman, M. L. et al. An orally bioavailable parthenolide analog selectively eradicates acute myelogenous leukemia stem and progenitor cells. Blood 110, 4427-4435 (2007).
21. Löpez-Franco, O. et al. Nuclear factor-kappa B inhibitors as potential novel anti-inflammatory agents for the treatment of immune glomerulonephritis. Am. J. Pathol. 161, 1497-1505 (2002).
22. Wiggins, J. E. Aging in the glomerulus. J. Gerontol. A Biol. Sci. Med. Sci. 67, 1358-1364 (2012).
23. Brähler, S. et al. Intrinsic proinflammatory signaling in podocytes contributes to podocyte damage and prolonged proteinuria. Am. J. Physiol. Renal Physiol. 303, F1473-1485 (2012).
24. Zhao, X., Hsu, K.-S., Lim, J. H., Bruggeman, L. A. & Kao, H.-Y. α-Actinin 4 potentiates nuclear factor κ-light-chain-enhancer of activated B-cell (NF-κB) activity in podocytes independent of its cytoplasmic actin binding function. J. Biol. Chem. 290, 338-349 (2015).
25. Wei, M., Li, Z., Xiao, L. & Yang, Z. Effects of ROS-relative NF-κB signaling on high glucose-induced TLR4 and MCP-1 expression in podocyte injury. Mol. Immunol. 68, 261-271 (2015).
26. Bao, W. et al. Toll-like Receptor 9 Can be Activated by Endogenous Mitochondrial DNA to Induce Podocyte Apoptosis. Sci Rep 6, 22579 (2016).
27. Liu, Q. et al. Inhibition of NF-κB Reduces Renal Inflammation and Expression of PEPCK in Type 2 Diabetic Mice. Inflammation 41, 2018-2029 (2018).
28. Nakshatri, H. et al. NF-κB-dependent and -independent epigenetic modulation using the novel anti-cancer agent DMAPT. Cell Death Dis 6, e1608 (2015).
29. Hadden, M. J. & Advani, A. Histone Deacetylase Inhibitors and Diabetic Kidney Disease. Int J Mol Sci 19, (2018).
30. Curry, E. A. et al. Phase I dose escalation trial of feverfew with standardized doses of parthenolide in patients with cancer. Invest New Drugs 22, 299-305 (2004).
31. Liu, X. et al. Isolating glomeruli from mice: A practical approach for beginners. Exp Ther Med 5, 1322-1326 (2013).
32. Takemoto, M. et al. A new method for large scale isolation of kidney glomeruli from mice. Am. J. Pathol. 161, 799-805 (2002).
33. Klein, J. et al. Urinary peptidomics provides a noninvasive humanized readout of diabetic nephropathy in mice. Kidney Int. 90, 1045-1055 (2016).
34. Reimand, J. et al. Pathway enrichment analysis and visualization of omics data using g:Profiler, GSEA, Cytoscape and EnrichmentMap. Nat Protoc 14, 482-517 (2019).
35. Subramanian, A. et al. A Next Generation Connectivity Map: L1000 Platform and the First 1,000,000 Profiles. Cell 171, 1437-1452.e17 (2017).

1. A method of treating chronic kidney disease (CKD) in patient in need thereof comprising administering to the patient a therapeutically effective amount of Dimethylaminoparthenolide (DMAPT).

2. The method according to claim 1 wherein the CKD is a disease selected from the group consisting of nephropathy, glomerulonephritis, interstitial nephritis, lupus nephritis, idiopathic nephrotic syndrome (INS), obstructive uropathy, polycystic kidney disease, cardiovascular diseases, hypertension, diabetes, and kidney graft rejection.

3. The method according to claim 1 wherein the CKD is a diabetic kidney disease (DKD).

4. The method according to claim 3 wherein the patient with DKD has type 1 diabetes.

5. The method according to claim 3 wherein the patient with DKD has type 2 diabetes.

6. The method according to claim 1 wherein the CKD is a progressive CKD after a partial nephrectomy.

7. The method according to claim 1 wherein the CKD is a focal segmental glomerulosclerosis (FSGS).

8. The method according to claim 1 wherein the patient has a vascular calcification.

9. The method according to claim 8 wherein the vascular calcification is an intimal calcification or a medial calcification.

10. The method according to claim 1 wherein the DMAPT is combined with a nephroprotective treatment.

11. The method according to claim 10 wherein the nephroprotective treatment is sodium-glucose cotransporter 2 inhibitor (SGLT2i).

12. The method according to claim 10 wherein the nephroprotective treatment is a renin angiotensin aldosterone system inhibitor (RAASi).

13. The method according to claim 12 wherein the RAASi is an angio-converting enzyme inhibitors (ACEi).

14. The method according to claim 13 wherein the ACEi is ramipril.

15. The method according to claim 12 wherein the RAASi is an angiotensin-receptor blockers (ARBs).

16. The method according to claim 10 wherein the nephroprotective treatment is an anti-endothelin-1 receptor.

17. The method according to claim 2 wherein
the nephropathy is membranous nephropathy (MN), diabetic nephropathy or hypertensive nephropathy,
the glomerulonephritis is membranous glomerulonephritis or membranoproliferative glomerulonephritis (MPGN),
the INS is minimal change nephrotic syndrome (MCNS) or focal segmental glomerulosclerosis (FSGS),
the polycystic kidney disease is Autosomal Dominant Polycystic Kidney Disease (ADPKD) or Autosomal Recessive Polycystic Kidney Disease (ARPKD),
the diabetes is diabetic kidney disease, and
the kidney graft rejection is acute or chronic kidney rejection.

18. The method of claim 17, wherein the MPGN is rapidly progressive glomerulonephritis (RPGN).
